(19) United States
(12) Patent Application Publication
(10) Pub. No.: US 2021/0082317 A1
(43) Pub. Date: Mar. 18, 2021

(54) Multiple Format Instructional Display Matrix Including Real Time Input

(71) Applicant: University of South Carolina, Columbia, SC (US)

(72) Inventors: Richard Hoppmann, Columbia, SC (US); Toufic Haddad, Columbia, SC (US); Sean David Lee, Lexington, SC (US); Christopher David Lee, Lexington, SC (US)

(73) Assignee: University of South Carolina, Columbia, SC (US)

(21) Appl. No.: 16/961,294

(22) PCT Filed: Jan. 18, 2019
(86) PCT No.: PCT/US2019/014179

§ 371 Date: Jul. 10, 2020

Related U.S. Application Data
(60) Provisional application No. 62/619,154, filed on Jan. 19, 2018.

(51) Int. Cl. G09B 23/28; G06F 3/14; G09B 5/06; G09B 5/02; G16H 30/40; G06N 5/02

(52) U.S. Cl. CPC G09B 23/286; G06F 3/1454; G09B 5/065; G06F 40/58; G16H 30/40; G06N 5/02; G09B 5/02

(57) ABSTRACT

A real-time, multiple clinical input system that allows independent control of each input data set, synchronization of data, display of data on a single multi-section matrix screen, and also allows for recording clinical data.

### BACKGROUND OF THE INVENTION

### 1) Field of the Invention

The present disclosure relates to a real-time, multiple clinical input. instructional system that allows independent control of each input data set, synchronization and display of data on a single multi-section matrix screen, and allows an instructor to easily record clinical data.

### 2) Description of Related Art

In the United States alone, there are 172 medical schools, over 3000 nursing schools, over 250 physician assistant programs, over 350 simulation centers, and over 300 teaching hospitals. Medical education has been criticized in recent years for having changed little despite advances in medical and teaching technology, the understanding of learning theory, and the importance of patient-centered healthcare. Medical educators concur that instructional innovations are long overdue and much needed. The U.S. health-care system is rapidly becoming ever more data-driven, evidence-based, patient-centered and value-oriented. But for reasons having to do with tradition, accreditation concerns and preparing students for national board exams, the designers of medical-school curricula have been slow to shift educational focus. From a learning perspective, the value of interactive learning has been emphasized across virtually all educational systems, and the integration of curricular content is becoming a priority for medical education.

The Reality Instructional Matrix System (“RIMS”) of the current disclosure addresses many of the concerns in medical education today. This instructional system utilizes and integrates the latest in medical technology such as portable ultrasound, digital clinical devices such as electronic stethoscopes, portable ECG, smart phones, and digital cameras. It also is designed to easily add new device data as they are introduced into the market. RIMS allows for interactive learning and the integration of content from a variety of inputs to include anatomy, physiology, pathology and diagnosis of health and disease in a way that has never been available in medical education. There are presently no real-time multiple clinical input instructional systems on the market that allow independent control of each input data set, synchronization of data, display of data on a single multi-section matrix screen, and allows the instructor to easily record clinical data.

There are numerous simulation systems that use manikins to educate but none of these use real-time patient input nor do they have the display capabilities of the current disclosure. Current medical simulation companies include: SIMULAB https://www.simulab.com/?gclid=CjwKCAiwmaHPBRBQEiwAOvbR8_ZEf2Y4M7gi7 ZDDDapuV-H07PhmKAAW-cqtl9143QldZa9vsA4b_RoCJ10QAvD_BwE: VATA Anatomical Healthcare Models https://www.vatainc.com/?gclid=CjwKCAiwmHPBRBQEiwAOvbR8w7dCDNIciF8fr JI_plewq2jWMQ4nhfGSGM7li2C67Kk2XiOOWfySxoCahsQAvD_BwE; SIMStation https://www.level3healthcare.com/simstation/?gclid=CjwKCAiwmaHPBRBQEiwAO vbR8ynf5TnSZiKahJOcamfcxx36df-GHCRng4njPHklE0FqG4WJiggEnhoCfvEQAvD_BwE; 4. MSC Med Simulation Company http://www.medsimulation.com/; 3D Systems—Simbionix http://simbionix.com/; and CAE Healthcare https://caehealthcare.com/; Limbs and Things https://limbsandthings.com/us/.

Various patient monitoring services are also available, but these, too, lack the functionality of the current disclosure. Patient Monitoring: Philips Patient Monitoring—https://www.usa.philips.com/healthcare/solutions/patient-monitoring?origin=7_700000001652071_71700000027999543_58700003605308150_43700028101851055; and icumedical—http://www.icumed.com/nroducts/critical-care/hemodynamic-monitoring-systems.aspx.

RIMS would give a distinct advantage to medical education companies that compete with manikin simulation companies. There is simply no manikin simulator experience that can match interacting with a live patient or model and analyzing clinical data in real-time. In a profession like medicine, in which interacting with another individual is critical to the quality of service provided, being able to learn and practice with real patients or live models will likely become state-of-the-art training in the health professions. In addition, RIMS will give established manikin simulation companies an advantage if they offer it as a complement to the traditional manikin simulation experience. The traditional manikin simulation companies can also enhance their own product by incorporating the RIMS multi-format matrix display solution and the education recording suite into their simulators to gain a market advantage.

At present there are no patient monitoring systems that allow the degree of control over the clinical input display format as that of RIMS as explained infra. Accordingly, it is an object of the present invention to provide RIMS display capabilities will provide better educational experiences and patient care in true healthcare delivery settings.

### SUMMARY OF THE INVENTION

In a first embodiment, the current disclosure provides A real time, multiple input clinical display system. The system includes a multi-sector matrix screen that has multiple screen sectors. The multi-sector matrix screen is configured to display multiple data inputs on or within the multiple screen sectors simultaneously. Further, the multiple data inputs comprise real time patient diagnostic information. Also, the multiple data inputs can comprise previously recorded or web-based material. Yet still, the display size of data inputs shown within the multiple screen sectors is variable. Still further, at least one of the multiple medical inputs may be input via voice-command. Further, yet the multiple data inputs include, at least, a side-by-side comparison of real-time ultrasound scanning images and an instruction input. Yet further, the instructional input is an instruction video displaying property ultrasound scanning technique. Still again, data inputs from extraneous devices may be mirrored on at least one screen sector of the multi-sector matrix. Yet still, the multiple data inputs include real time medical diagnostic analysis of a patient shown simultaneously with previously obtained diagnostic information of the patient. Further yet, display speed of the multiple data inputs on the multiple screen sectors is variable. Moreover, the multiple data inputs displayed are recorded by the system. Yet again, the system includes parametric speakers. Still yet again, control of a cursor associated with the display can be transferred from one user to another user. Yet still, an instructor's oral presentation of information is shown as scrolling text on the display. Again, the instructor's oral presentation is translated into at least one other language and this language is displayed as scrolling text on the display. Further still, artificial intelligence (AI) is utilized for image and clinical data interpretation of the multiple data inputs. Yet further, presentation of the AI clinical data interpretation on the display is inactivated to allow for analysis prior to the A interpretation. Yet still, audience responses are displayed in at least one multiple screen sector. Further, remote access to the display information is provided. Still yet, clinical data from two or more real time, synchronized inputs can be interpreted with or without AI. Further still, input and control of the display may be via remote control. Yet again, users may access a frequently asked questions directory via displaying the frequently asked questions directory on the display.

In a further embodiment, a system for clinical analysis is provided. The system includes a multi-sector matrix screen further comprising multiple screen sectors, digital recording means to record all information shown on the multiple screen sectors, at least one parametric speaker, at least one camera, at least one sound recording device, and the multi-sector matrix screen is configured to display multiple data inputs on the multiple screen sectors simultaneously.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows a teaching method of one embodiment of the current disclosure.

FIG. 2 shows a teaching system of one embodiment of the current disclosure.

FIG. 3 shows a picture of one embodiment of a multi-sector display of the current disclosure.

FIG. 4 shows a picture of two simultaneous synchronized ultrasounds of difference areas of the body as one embodiment of the current disclosure.

FIG. 5 shows a picture of mirroring an ultrasound image from a smart phone to RIMS as one embodiment of the current disclosure.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

The Reality Instructional Matrix System (RIMS) is a multiple format display solution (or matrix) which incorporates HDMI, WEB content and software into a single display solution. In one embodiment, custom designed software has been employed including, but not limited to: (1) an education suite for recording and playing back Medical Cases; (2) RIMS control suite—ensures all programs running in proper viewports, prevents system crashes from causing problems, and reboots the computer into update mode when updates are received; (3) Shutdown—prompts the user with a problems/feedback section they can fill out with any problems which will be sent directly to headquarters upon shutdown—ensures the proper shutdown and backup; (4) alclear —prevents startup problems after audacity crash. called by Audacity; (5) program_guardian—restarts crashed programs in proper window, called by rims_control_suite; (6) Audacity—call to start audacity, called by program_guardian); (7) hdmi—starts hdmi input feed. called by program guardian; (8) play_case—script to play recorded case; (9) record_case—script to record case; (10) rims_options_menu—menu to turn program_guardian on or off; (11) safelist—prevents unauthorized USBs from being mounted into the filesystem and used; (12) stscr—part of the startup sequence. called by system_start; (13) system_start—determines whether to start in normal or update mode; (14) firefox_start —call to start firefox. called by program guardian; (15) vm_cardio_start—call to start cardioperfect VM, called by program guardian; (16) vm_android1_start—call to start android1 vm for pulse oximeter. called by program guardian; (17) vm_android2_start—call to start android2 vm for blood pressure cuff, called by program guardian; (18) vm_win_airplay1_start—call to start windows vm for D-eye airplay. called by program guardian; (19) vm_win_airplay2_start—call to start windows vm for extra airplay, called by program guardian. Commercial software may also be used with the current disclosure including but not limited to: (1) VMware Workstation 14; (2) Audacity; (3) Welch Allyn Cardio Perfect; (4) VLC; (5) Firefox; (6) AirServer Universal; (7) pavucontrol; (8) SoftEther—used for site-to-site edge vpn routing, allowing remote access from headquarters; (9) G++− for compilation of C/C++ code; and (10) Visual Studio Code (coding IDE that operates out of folders. Further, the current disclosure may work with Google translate to enable multi-language functionality as well as with voice command software and/or voice recognition/dictations software such as DRAGONT™ to enable hands free or voice command capabilities. This enables RIMS to recognize voice commands to activate the system, move input to various sectors of the screen, download from other sources like the internet, and other command operations. Further, an instructor's oral presentation of information while using the system for instruction may be shown as scrolling text on the display. Even further, the oral presentation can be translated into at least one other language and this language is displayed as scrolling text on the display.

Further the current disclosure also has wireless mirroring capability to receive input from one or more digital devices such as smart phones and tablets via WIFI or Bluetooth technology to project onto various screen sectors. Mirroring material can include but would not be limited to diagrams, pictures of pathology, medical imaging such as X-rays, CT, MRI, coronary artery fluoroscopy, and ultrasound. Real-time data from clinical devices can also be mirrored such as ultrasound image, ECG recordings, pulmonary function tests results, and other real-time sources. Data can be mirrored directly to RIMS when possible or it can be sent to a smart device such as a phone and then mirrored to RIMS.

The system includes an education suite for recording and playback, as well as an Audio Analysis system. RIMS also recognizes voice commands to activate the system, move input to various sectors of the screen, download from other sources like the internet, and other command operations. Multiple external inputs can be synchronized on a single multi-sector screen with independent control of each sector as needed.

Utilizing real-time clinical input from patients/models that directly interact with the learners, a patient-centered approach to providing healthcare can be modeled, taught, practiced, and assessed at the bedside that is not possible with a simulation manikin. Additionally, these learning experiences can include many communication and technical skills applicable to a variety of healthcare providers from nurses, physician assistants, medics and physicians which makes this invention well-suited to team-based learning and inter-professional training.

The technology of RIMS, including real-time demonstrations can also be used for larger groups of learners including in an auditorium setting with RIMS projection onto an appropriate sized screen. RIMS also has the capability for real-time speaker/instructor translation into one or more languages for a multi-national audience. The real-time translation scrolls across one of the screen sectors.

In one aspect, one sector of RIMS can receive and display input from an audience response system such as “Poll Everywhere” (https://www.polleverywhere.com/?ref=PIW0gbZ&campaignid=1624296850&adgro upid=63462208002&keyword=poll%20evervwhere&gclid=Cj0KCQiAiZLhBRCAARI sAFHWpbHY4gSiYITa8AQ8w4KRd_0b7qst7leMYok-wYbT3EQm2GzQIrFn368aAkWtEALw_wcB) for small or large group interactive learning. For example, the audience could rate the quality of an ultrasound image, identify pathology in a slide, or vote on what should be done clinically for the patient next. The group response can be displayed in one RIMS sector and discussed. In addition, serious games for learning can be displayed on RIMS, such as SEPTRIS (http://med.stanford.edu/septris/game/SeptrisTitle.html), for audience interaction.

Having the ability to record the real-time clinical input also provides opportunities for review and reflection with learners as well as development of additional learning material for self-directed independent learning. These are important considerations in medical education, especially in today's competency-based medical education model.

While the learner is scanning with ultrasound in real-time the scanning loops are being recorded by RIMS. The recorded loops can be stopped and rewound by the instructor with a remote device. This will allow the instructor to review the rewound segment with the group of learners to enhance teaching. If RIMS is being used for remote medical consultation this feature will allow enhanced consultation as both parties can review the captured segment for more accurate feedback, diagnosis, and patient management.

For ease of use, especially for new users or infrequent users, RIMS has a frequently asked questions (FAQ) feature for the user to search in real-time for answers to common RIMS operational/functionality questions.

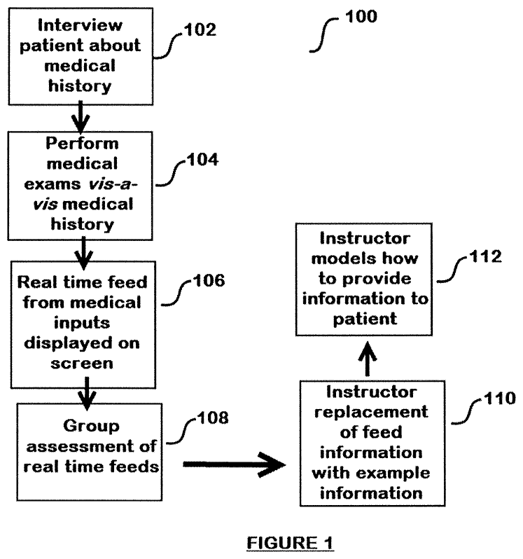

As an example of how the system can be used, consider when students are studying hypertension and its effect on the heart, a RIMS session can be scheduled with a patient known to have high blood pressure. With respect to FIG. 1, an example teaching scenario 100 is shown. At step 102, the instructor and students can interview the learners about his/her medical problems. At step 104, the instructor and learners then perform a heart ultrasound (ECHO), an EKG, listen to the heart sounds with an electronic stethoscope, and measure blood pressure either manually or by an attached automated blood pressure cuff, all of which may be recorded and stored. At step 106, real-time feed from all four inputs would be displayed on the RIMS screen for viewing and discussion. The group could assess, at step 108, the ultrasound display for evidence of left ventricular hypertrophy or thickening of the heart muscle of the left ventricular that can be seen in patients with chronic hypertension, the ECG would likewise be evaluated for hypertension related changes, and all participants could listen for abnormal heart sounds that can be heard in patients with hypertension or heart failure such as S3 or S4 heart sounds which would also be displayed graphically. The patient's blood pressure could also be displayed.

During the session, at step 110, the instructor may replace one of the real-time inputs with recorded examples of normal or pathological heart findings for comparison with the patient's. At the end of the session, at step 112, the instructor could model for the group of learners how best to present the findings to the patient or the instructor could ask one of the learners to present the findings to the patient and then provide feedback to the learner and others participating in the session.

The education suite of the system will allow the instructor to record particularly good examples of the clinical data displayed to review with the learners or to develop educational material to be incorporated into future learning sessions. In addition, video loops such as those recorded of ultrasound of the heart may be shown at full speed or at ½ or ¼ speed for the learner to better visualize the anatomical and physiological changes of a dynamic heart. Further, learners can point out structures on the screen with a light/laser pointer or can take control of the RIMS cursor with a smart phone app. Further, the speed of the data, videos, diagnostic information shown on the system is variable and may be sped up or slowed down as the user requires. Further, a user may temporarily halt or “pause” the information shown on the display and can rewind or fast-forward same. This would be applicable only to the information contained within the system and would not influence an actual ultrasound or other procedure being performed on a patient.

For more of a self-directed learning approach, a recorded video of how to perform a particular ultrasound scan could be shown on one of the sectors while the learner in real-time scans the patient with ultrasound following the instructions in the tutorial video. The tutorial can be slowed down (i.e., half-time) to more easily comprehend new material or sped up (i.e., double time) for more efficient use of learning time for material already well known. The tutorial video can also be stopped at key points so the learner can try to replicate the ideal ultrasound images in the tutorial. Thus, a “target” or “ideal” ultrasound image could be displayed on one sector as the learner tries to match it on the adjacent sector of the screen while actively ultrasound scanning the patient/model.

In a further embodiment, the system may be equipped with laser or parametric speakers to limit the sound produced from the system to an area encompassing only the group of learners at the particular RIMS station. This will allow multiple RIMS stations to be running in a room at one time without overlapping sound from each station. In one embodiment, directional sound may be employed. Directional sound is a technology that concentrates acoustic energy into a narrow beam so that it can be projected to a discrete area, much as a spotlight focuses light. Focused in this manner, sound waves behave in a manner somewhat resembling the coherence of light waves in a laser. When a sound beam is aimed at a listener, that person senses the sound as if it is coming from a headset or from “inside the head.” When the listener steps out of the beam, or when the beam is aimed in a different direction, the sound disappears completely. Several techniques are available to accomplish this. For instance, the ACOUSPADE™ hyper directional speaker, from Ultrasonic Audio Technologies, can deliver a narrow beam of sound to a desired area while preserving silence around it, or allowing the co-existence of different sounds in the same space without mixing or interfering. The audio-beam created by ACOUSPADE can cut through noisy environments and deliver a headphone-like experience for the listener.

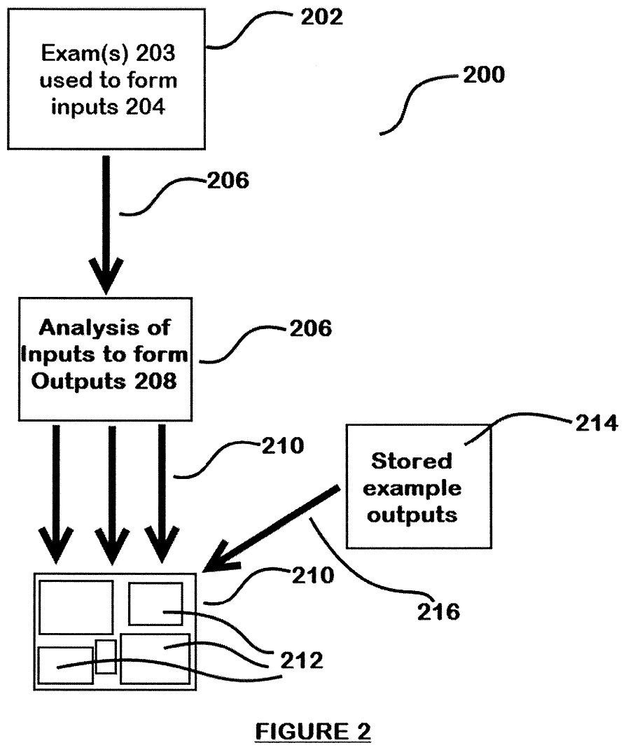

FIG. 2 illustrates schematically a further teaching method and system 200 of the current disclosure. Here, at step 202 a patient would undergo a physical exam 203, such as for purposes of example only and not intended to be limiting, a cardiac exam wherein the patient's vital signs, including blood pressure. physical characteristics, verbal responses to questions. etc., are taken and recorded. This step may also include the use of various medical devices, such as heart monitors, electronic blood pressure measuring devices, etc., that may be used to analyze and record the patient's physical condition. The results for inputs 204 from exam 203 are then transmitted for analysis at step 206. Analysis may be performed by Artificial IntelligenceMachine Learning medical software to analyze the data and propose a diagnosis. Examples may include IBM Watson (https://www.ibm.com/watson/health), Isabel (https://www.isabelhealthcare.com/), and Human Dx (https://www.humandx.org-). For self-directed learning, the learners using RIMS would analyze the clinical data themselves and then activate the artificial intelligence (AI) to analyze the data. The learners could then compare their analysis and diagnosis with that of the AI diagnosis and discuss how they were alike or different. AI would also be used in real-medical situations such as in hospitals. clinics, or even tele-health remote settings. Further, remote learning opportunities are promoted as onsite cameras will allow visualization of all parties including patients, if present, as well as instruction of the learner in manipulation of the clinical device such as an ultrasound probe to obtain the most accurate clinical data.

Analysis of inputs 204, at step 206, then forms outputs 208. Outputs 208, may comprise data, flow charts, readouts (e.g., EKG readouts, blood pressure reports, etc.), statistical information, comparative data, etc., as known to those of skill in medical arts, that displays the information collected during exam 203 and used to form inputs 204. In embodiment, the output may be [HDMI/H.264; HDMI can also be DVI, Component, Composite, or any other video format. At step 210, outputs 208 may be transferred to multi-sector display 210. Outputs 208 may be delivered as data, such as a digital bitstream or a digitized analog signal over a point-to-point or point-to-multipoint communication channel.

ECG input can be from a single lead ECG recording or multiple leads up to the traditional 12 leads and beyond. RIMS does not receive each individual lead directly. Input from each lead is first processed by the peripheral ECG device and the results are sent to RIMS for the composite ECG display and interpretation.

Examples of such channels include, but are not limited to, copper wires, optical fibers, wireless communication channels, storage media and computer buses. The data may be represented as an electromagnetic signal, such as an electrical voltage, radiowave, microwave, or infrared signal. Analog transmission may send the data as a continuous signal which varies in amplitude, phase, or some other property in proportion to that of a variable. The messages are either represented by a sequence of pulses by means of a line code (baseband transmission), or by a limited set of continuously varying wave forms (passband transmission), using a digital modulation method. The passband modulation and corresponding demodulation (also known as detection) is carried out by modem equipment. According to the most common definition of digital signal, both baseband and passband signals representing bit-streams are considered as digital transmission, while an alternative definition only considers the baseband signal as digital, and passband transmission of digital data as a form of digital-to-analog conversion. Data transmitted may be digital messages originating from a data source, for example a computer or a keyboard. It may also be an analog signal such as a phone call or a video signal, digitized into a bit-stream for example using pulse-code modulation (PCM) or more advanced source coding (analog-to-digital conversion and data compression) schemes. This source coding and decoding is carried out by codec equipment.

Multi-sector display 210 receives outputs 208 and converts these to visual displays 212. Conversion of outputs 208 from one data form to another may be accomplished via a computer environment. For example, computer hardware such as H.264 Encoder; HDMI on-board/or PCI expansion may convert the data using a typical software platform. Data conversions may be as simple as the conversion of a text file from one character encoding system to another; or more complex, such as the conversion of office file formats, or the conversion of image and audio file formats. In some cases, a computer program may recognize several data file formats at the data input stage and be capable of storing the output data in a number of different formats.

Multi-sector display 210 may show visual displays 212 in a wide variety of informational formats. This includes, but is not limited to, quantitative displays, which provide information about the numerical value or quantitative value of outputs 208. The quantitative display may be either dynamic (i.e. changing with time such as pressure or temperature) or static. Multi-sector display 210 may also provide qualitative displays that provide information about a limited number of discrete states of some variable, such as blood pressure, heart rate, blood volume, blood glucose, pulmonary function tests, temperature, etc. These displays provide qualitative information, i.e. instantaneous (in most cases approximate), values of certain continuously altering/changing variables such as pressure, temperature, which may provide the general trend of change for the qualitative information.

Quantitative values and displays can come from standard equipment or newer technology such as smart phones, smart watches, fitbits, and other medical “wearables.” In addition, one or more sectors can be used to display historical information from the stored medical record of the patient or listed by voice recognition/dictation software such as DRAGON™ from the RIMS instructor or healthcare provider that is obtained while interviewing the patient during the encounter. These can include patient reported symptoms, past and present medications, previous test results, family history, and other important clinical information. This data will also be available for artificial intelligence analytics for more accurate clinical diagnoses and as an educational tool as well. This allows for a host of functionality as a user may direct the system to search for and display information contained within the system. For example, a user could instruct the system to “download the heart ultrasound instructional video to sector 2 of the screen.”

Multi-sector display 210 may also provide pictorial displays, such as photographs, television screen radarscope, flow diagrams, body schematics, etc. Multi-sector display 210 may also provide auditory displays, such as tones, frequencies, sounds created by devices used to analyze the patient, etc. In further embodiments, multi-sector display 210 may also be associated with other devices in order to provide tactile information to the user of the multi-sector display, such as a refreshable braille display or braille terminal.

Multi-sector display 210 may comprise, but is not limited to, Eidophor Electroluminescent display (ELD), Electronic paper E Ink Gyricon Light emitting diode display (LED), Cathode ray tube (CRT) (Monoscope), Liquid-crystal display (LCD) TFT TN LED Blue Phase IPS), Plasma display panel (PDP) (ALiS), Digital Light Processing (DLP), Liquid crystal on silicon (LCoS), Organic light-emitting diode (OLED) (AMOLED), Organic light-emitting transistor (OLET), Surface-conduction electron-emitter display (SED), Field emission display (FED), Laser TV (Quantum dot) (Liquid crystal), MEMS display (IMoD TMOS DMS), Quantum dot display (QD-LED), Ferro liquid crystal display (FLCD), Thick-film dielectric electroluminescent technology (TDEL), Telescopic pixel display (TPD), and Laser-powered phosphor display (LPD), or combination of the above. Multi-sector display 210 may also include 3D display technologies, such as Stereoscopic, Autostereoscopic, Multiscopic, Hologram Holographic display Computer-generated holography), Volumetric, Musion Eyeliner, and Fog display.

In a further embodiment, multi-sector display 210 may include sectors on the display, see FIG. 2, that may be enlarged for clarification of the visual display and facilitate more focused instruction of a particular aspect of the subject matter. Real-time input to each sector can include but are not limited to ultrasound video of the heart being performed by the instructor or learner, an electrocardiogram of the heart (ECG), heart sounds recorded from an electronic stethoscope with audio analysis graphically displayed, blood pressure readings, and blood glucose levels from the patient or model. Displayed material can be swapped out for additional instructional material such as previously recorded 3D anatomical images, instructional videos on ultrasound scanning, additional clinical input such as digital retinal images of the eye, pictures of skin lesions taken with a digital camera, pulmonary function tests results, and any variety of Web content. Further, the system may be compatible with smartphones and tablets to allow mirroring of input from these devices to be shown on the display.

In addition, referring again to FIG. 2, a catalog of stored example outputs 214 may also be associated with Multi-Sector Display 210 and delivered to Multi-Sector Display 210 at step 216. This would allow an instructor to compare the stored example outputs 214 with outputs 208 generated from the patient for instructional, comparative, or other purposes. Stored example outputs 214 may be shown in conjunction with or supplant the data shown by Multi-Sector Display 210, such as shown in outline, a side-by-side comparison, etc. Multi-Sector Display 210 may also provide two-way communication between the instructional setting and the patient from whom inputs 204 were received in order to allow real time instruction and to allow for obtaining additional, real-time input from the patient. This may be accomplished using a computer network to have two-way communication by having computers exchange data such as through wired and wireless interconnects. The system may also be configured to allow for remote, interactive educational conferencing across significant distances. RIMS cameras may also allow real-time educational as well as medical consultation communication (tele-health). Control of certain RIMS functions such as that of a cursor to point out specific findings or display new material can be transferred to remote viewers if they have RIMS or by way of a remote downloadable application.

Patients with genuine pathology and their corresponding clinical history can be used in these learning experiences or healthy models can be trained to describe a clinical history consistent with the disease being studied. With healthy models recorded, pathological findings, such as stored example outputs 214, may be used during the sessions to enhance the learning experience.

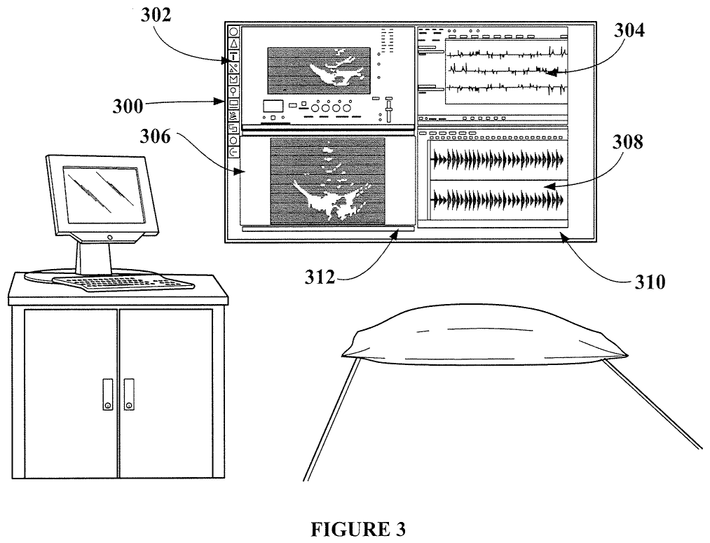

FIG. 3 shows a picture of one embodiment of a multi-sector display 300. In this embodiment, several different informational displays are shown, such as ultrasound 302. graphic 304. which may be for purpose of example only an EKG readout, instructional video 306, which for purposes of example only may be a video of how to ultrasound scan the heart, as well as a graphic of heart sounds 308 from an electronic stethoscope of the patient/actor being analyzed for the instructional session. As discussed previously, all feeds may be real time but may also comprise pre-recorded information that may be displayed via multi-sector display 300. The number of multi-sectors displayed does not have to be limited to four. Fewer or more sectors can be displayed as a function of the data to be displayed, the size of the screen, and the processing power of the system. Further, the size of the images displayed is variable and may be enlarged or shrunk as the user prefers as known to those of skill in the art. Display 300 may also include a camera 310 to enable transmission of audience video as well as the instructor's guidance to attendees at remote locations. Further, a speaker 312, such as a parametric speaker. will allow the audience to hear the instructor but only project the sound to a small area so as not to disturb surrounding patients, educators, etc.

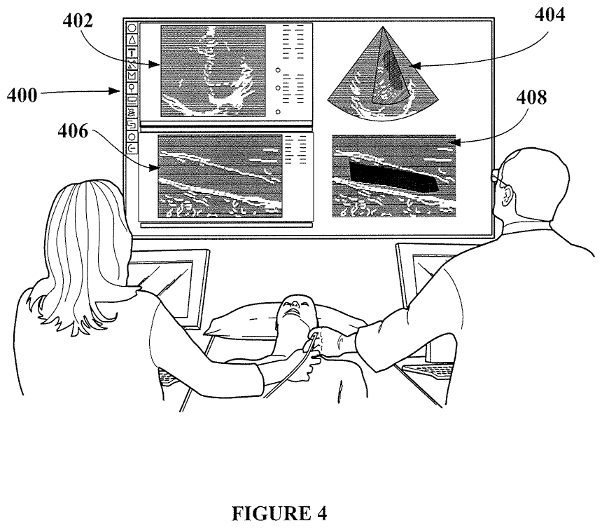

When two of the sectors are used for real-time independent but synchronous ultrasound scanning, unique clinical information and teaching opportunities will be possible for the first time and could have wide reaching implications for patient care and education. For example, FIG. 4 shows a picture of one embodiment of synchronous scanning in multi-sector display 400, an ultrasound of the heart without color Doppler 402 and an ultrasound of the heart with color Doppler 404 synchronized with ultrasound of the blood vessels in the neck (the carotid artery and the internal jugular vein) without color Doppler 406 and with color Doppler 408. This combination would yield simultaneous information on the cardiovascular system that is not presently available and will aid in the assessment of heart function and vascular circulation with significant implications for diagnosis and management of patients as well as advance our understanding of cardiovascular diseases. This new combination of ultrasound scanning data will also be available for artificial intelligent analytics and deep learning.

This RIMS offers many advantages for instructors and learners. For the first time ever, important clinical information can be simultaneously displayed and synchronized such as visualization of a beating heart with ultrasound while listening to the heart sounds from that particular patient. Combining this information with real-time ECG reading and additional clinical information, or supplemental recorded educational material, will create an extraordinary and unique learning experience. In addition, RIMS has been designed with the flexibility to accept other types of digital data that may become available in the future.

The current disclosure provides immediate improvements over existing teaching modules. A RIMS session could include active learning of clinical skills such as performing ultrasound and interpretation of a variety of clinical data as it is typically done in diagnosing and managing medical conditions. The source of the clinical data would be real patients or trained models and not simulation manikins. Despite the technical sophistication of instructional manikins, the manikin experience still simply falls short of a true human-to-human learning experience that is so important to the development of good patient-healthcare provider interaction.

Real patients and trained models are also much better than manikins for teaching important physical examination skills like palpation, auscultation, and percussion (tapping on the surface of the body to assess structures below the skin like the liver or lung). With the live model, the auditory (electronic stethoscope) and the visual (ultrasound image) feedback the RIMS provides can significantly enhance learning auscultation of the heart. In a patient with abdominal pain, the skill of palpating the liver and gallbladder for tenderness is a critical component of the physical examination. When learning this skill, ultrasound can be used to visualize the liver and gallbladder as they come further down into the abdomen with a deep respiratory inspiration. With ultrasound the learner can see the liver and gallbladder as they reach the area where the learner is pressing into the abdomen and touches his/her fingertips. This immediate visual and tactile feedback can enhance the learning of exactly where and what a liver and gallbladder should feel like.

Having a live subject creates more realistic experiences for learners to develop the skills of interacting professionally and effectively with patients. It is critical that healthcare providers develop these communication skills, learn to be attentive to patient needs, and treat them with dignity and respect at all times. Learners need to be particularly sensitive to patient needs while performing procedures such as only exposing those areas of the body that need to be exposed while performing an ultrasound examination. Learners also need to develop good patient education skills and how to work as part of an inter-professional healthcare team.

The RIMS can be used in the classroom or small group didactic sessions without a live model or patient. Medical cases that rely on multiple clinical data points to understand the disease process and make clinical decisions can be effectively presented with the RIMS. Recorded data, including data recorded from previous live patient sessions, can be used. In addition, RIMS may be adapted to a laptop and other portable devices with the same functionality with recorded and downloadable educational material for synchronous and asynchronous e-learning.

Additionally, RIMS may also be used in true medical settings that rely on monitoring continuous real-time multiple clinical data such as the intensive care unit of a hospital and other healthcare delivery settings. While maintaining its capability to teach learners in these settings, RIMS would also be used by the medical staff in monitoring and managing patients in real-time to improve the quality of healthcare provided.

This Reality Instructional Matrix System (RIMS) for teaching health professionals receives live clinical input from patients or live models. Input data can include but would not be limited to ultrasound images and videos (ECHO), electrocardiogram (ECG), and heart sounds from an electronic stethoscope. The input would be displayed on a single screen divided into multiple sectors for simultaneous viewing. These individual sectors can be independently controlled by the instructor and can be synchronized if necessary as with watching a beating heart on ultrasound and listening to the corresponding heart sounds. Additional information including diagrams, graphs, and videos can be included in one or more sectors to further explain the anatomy, physiology, or the disease process of the patient. This real-time, interactive, matrix display form of instruction is not presently available with live subjects and will greatly enhance the learning experience of medicine and other multi-faceted subject matter when compared to the presently used methods, including simulation manikins. The system can also be used with recorded materials only and not live patient input. RIMS can also be used in true medical settings such as the intensive care unit as both an instructional system and a patient care monitoring system.

In addition the RIMS system may be instrumental in use with developing tele-health systems given the real-time diagnosis and reference capabilities provided by the system. Moreover, the system may be used on-site at hospitals, clinics, emergency situations, etc., to provide real time medical care and monitoring for intensive care units, emergency rooms, operating rooms, etc. As an example, and not intended to be limiting, a healthcare provider in a rural area of a state without local access to medical specialists like cardiologists or radiologists could have a RIMS in their clinic, and once taught how to use the system, they could get real-time remote consultation with a specialist also using RIMS. Together they could see a patient with a history of chest pain using the RIMS cameras and interview the patient as well as review the real-time ECG, blood pressure, and ultrasound of the heart and lungs. Real-time video camera input can be displayed on one or more sectors for face-to-face communication/observation of the ultrasound probe or other device position remotely to instruct the health care provider in using the device while watching the screen together. Portable cameras can be attached to RIMS, the wall, a portable stand, or the ceiling.

In fact, the specialist could instruct the rural healthcare provider in obtaining the ultrasound image in real-time as both viewed the ultrasound image on the screen as well as the position of the ultrasound probe on the patient's chest. The rural healthcare provider could be a primary care physician, a nurse practitioner, a physician assistant, or another non-physician provider. With RIMS, the session could also be recorded for later review of the patient encounter and become part of the patient's record. In addition, with the artificial intelligence of RIMS, the rural provider would have a resource to assist with patient diagnoses even if a specialist was not available remotely. Thus, RIMS could have a significant impact on improving healthcare in those areas will limited healthcare access and specialty physicians.

In addition to teaching healthcare professions, RIMS could be used to teach life sciences across all levels of education, including primary and secondary schools, colleges, and universities. RIMS could also be adapted for use in teaching non-medical topics that would benefit from the integration and simultaneous presentation of multi-media material such as engineering, physical sciences, and aviation/aeronautics. Moreover, used as a patient monitor, use of RIMS may include all hospitals settings and many other healthcare delivery settings as well.

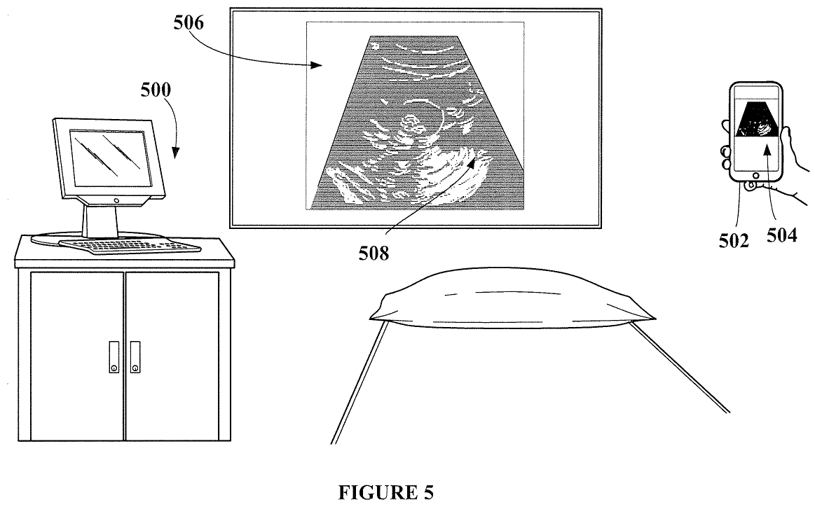

FIG. 5 shows a system 500 of the current disclosure wherein an image, ultrasound results or other data 502, here an ultrasound, shown on a handheld or other device 504, here a cell phone, is mirrored from device 504 to monitor 506 of the current disclosure and shown as image 508 on monitor 506. Mirroring may be accomplished via proprietary wireless protocol suites, such as AIRPLAY™, ROKU™, or applications such as MIRROR BETA, or dongles such as CHROMECAST™, as known to those of skill in the art.

The system of the current disclosure is very versatile. RIMS consists of multiple simultaneous and synchronized real-time medical data input that can be viewed and analyzed on a multi-sector matrix screen for enhanced medical education and patient care. In one instance, four screen sectors provide great flexibility for instruction without overwhelming the learner with input. However, RIMS can consist of fewer or more screen sectors as a function of the size of the screen, the quantity and type of display for each sector, and the processing power of the system. Previously recorded and web-based material can also be displayed in matrix sectors to complement the real-time clinical input. The instructor can also add important patient information such as patient reported symptoms or medication being taken to one of the RIMS sectors by way of voice recognition and dictation software. Individual screen sectors can be enlarged for detailed viewing of the sector material. RIMS allows unique ultrasound instruction with a side-by-side sector comparison of the learner's real-time ultrasound scanning images with an instructional “how to” scanning video with ideal ultrasound images that can serve as practice goals for the session. In addition to access to educational material stored within RIMS and internet access, RIMS will also have wireless mirroring capability of images, loops, videos, and other material from smart phones and other mirroring capable devices from participants. RIMS will allow side-by-side sector comparisons of previous patient data with newer or even real-time data obtained during the patient clinical encounter or teaching session. The RIMS instructor/learner will be able to slow down or speed up sector videos for educational purposes. RIMS can record the material displayed in the multiple sectors for review with learners and be used to create online and printed instructional resources for a wide variety of learners. Via a remote control, the instructor can stop a real-time scanning session of a learner such as ultrasound scanning and rewind the RIMS recorded segment for discussion This is stop/rewind control of the RIMS recording and not control over the individual real-time devices like an ultrasound system. RIMS can use voice commands like Google, Apple, and Amazon voice command systems. Each sector of the screen can be controlled by voice command such as “RIMS download the heart ultrasound instructional video to sector 2 of the screen.” RIMS can be equipped with parametric speakers to allow multiple training stations to be situated in an open space without one station's audio output from RIMS being heard at adjacent or more remote stations. Control of certain RIMS functions such as movement of the system's screen cursor can be transferred to non-instructor participants during RIMS sessions by a smart phone app allowing participants to point to anatomic structures or other material on the screen to ask questions or give responses to the instructor's questions. Real-time language translation of the instructor's voice will be available for screen sector display scrolling via translational software. Artificial Intelligence (AI) will be utilized for image and other clinical data interpretation for a more accurate diagnosis and as an educational tool. An “on/off” AI switch will allow the learner to first interpret the clinical data without AI, then with AI activation, a comparison can be made of the learner's diagnosis with the AI diagnosis as a form of learner self-assessment and instruction. RIMS will be able to receive wireless audience responses as with Poll Everywhere in one or more sectors for small and large group interactive presentations. RIMS will allow educational or “serious” medical games to be used in teaching small and large groups. RIMS can be used for large auditorium presentations with or without real-time demonstrations with clinical devices. Laptop, tablet, and smart phone versions of RIMS with primarily but not exclusively recorded material can be used for individual mobile education. RIMS allows remote real-time education and clinical consultation with viewing of the multiple screen sectors simultaneously by instructor/consultant and learner/consultee. For remote consultation and education, onsite cameras will allow visualization of all parties including patients, if present, as well as instruction of the learner in manipulation of the clinical device such as an ultrasound probe to obtain the most accurate clinical data. For remote consultation and education sessions, all RIMS functions will be available for use such as language translation, mirroring, and session recording. RIMS can be used as a patient diagnostic and monitoring system in medical practice settings such as emergency departments, intensive care units, outpatient settings, and other medical settings where monitoring of multiple clinical indicators improves patient care and healthcare professionals training. As seen in FIG. 4, RIMS can uniquely display and analyze real-time synchronization of two or more imaging studies such as an ultrasound of the heart and an ultrasound of the carotid artery in the neck. RIMS also has a searchable frequently asked questions (FAQ) feature to assist users in the operation of the system.

Further, the system of the current disclosure is built on a posix, LINNUX UBUNTU distribution. The current UBUNTU release used is 18.04 LTS. The current disclosure installs unity as it allows the most flexibility in video formatting. The screen can be divided into 4, six or nine screens each displaying in real time. Each of these screens will be referred to as a “Viewport”. The Viewport number is patterned the same as we read, from left to right, top to bottom. The system populates said windows upon startup with various programs used in medical evaluation. There is a windows as well as an android OREO virtual machine available for any app or software requiring either. The windows system also has AN AIRCAST, GOOGLECAST, MIRACAST server so users can cast their mobile devices to the system. The user system is controlled with a single executable written in C++, called “RimsSystem”.

RimsSystem is a multi-threaded software application that keeps track of system operation internally, and calls bash commands or other programs externally. The main function is split into three threads, each of which execute an initialization and a subsequent event loop to monitor interprocess variables and execute program functions. Each piece of software (AUDACITY, VMWARE, FIREFOX, etc., as known to those of skill in the art) has a software class associated with it. These classes have variables to keep track of the programs running status, and whether the user is focused on said program, as well as other possibilities. These Classes also have methods, such as StartQ, Stop( ), Clear( ), FocusOn( ), and SendWindowQ. Start and Stop both startup and shutdown the program. Clear is specific to Audacity, and clears any “lock files” that may have been generated from improper shutdown. FOCUSON tells RimsSystem to focus the user view on whatever Viewport the program is in. SendWindow(parameter) sends the program to whatever Viewport you designate in the parameter. Commands can be initiated by the user through individual call programs that are built into the user interface. These “call programs” use the BOOST library interprocess communication methods. When one of these programs are executed, they cause a memory location being monitored by the RimsSystem main( ) threads to become a logical TRUE, thus activating the associated function. Part of this execution is to return the interprocess memory location to logical FALSE before the loop completes its cycle. These commands include SystemStartup and SystemShutdown, Four(go to screen four), 4Screens, 9Screens, playcase, recordcase, gotobrowser, gotoultrasound, gotoheartsound, etc.

The RimsSystem executable has several special classes, not devoted to controlling software operation. One of these special classes is called RelativeResolution. Because of the way LINUX UBUNTU handles resolutions, it was found necessary to implement a means of controlling window size and location relatively instead of absolutely. Without this, different screens used with the system, be they 720 Progressive or 8K, could cause problems with windows being out of place or not showing whatsoever. The RelativeResolution class polls the current screen resolution horizontal and vertical, and then divides this number into percentages which are then used for absolute window placement. There is another special class called RSystem which handles the startup and shutdown of all processes controlled by the RimsSystem executable. The startup and shutdown sequences properly startup and shutdown all software, including saving any current work. In the case of the virtual machines, especially the Androids, it is imperative they be allowed to fully shut down, performing any required updates, before the main system begins to shut down. When the virtual machines are started, the main executable checks the configuration files for said machines and ensures all USB routing, network functionality, and display resolution will be correct. There is a script that runs after the Shutdown button is pressed that not only confirms they want to shut down, but provides opportunity to give feedback. This text is sent as a file named as the date, time and user through SSH over VPN to a central server. It is also optional to send a copy of the system's var/log folder every shutdown as well.

The RimsSystem executable has a system recording and playback suite that allows the user to record all on screen activity to a USB, as well as play any selected content. This Recorded content is stored on USB's that must be modified by us in order to work with the rims system. There is a script that runs at the end of the third thread's loop every 3000 us, that checks USB uuid's against a whitelist, and removes/unmounts any that are present and not supposed to be. It also sends text to the system warning popup that notifies the user that it must be removed. These usb's are part of a Paid-for educational content system, where educational content is sold to, and usable by only the machines associated with that account. This is done by simply assigning unique uuid's to be used for machines associated with a particular account, and sending the educational content on drives embedded with the particular uuid needed. To play any paid-for educational material, or third party content, the user presses “play content”, at which point the main executable prompts a headless vie to play the chosen file.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. A real time, multiple input clinical display system comprising:
a multi-sector matrix screen;
the multi-sector matrix screen having multiple screen sectors;
wherein the multi-sector matrix screen is configured to display multiple data inputs within the multiple screen sectors simultaneously.

2. The real time, multiple input display of claim 1, wherein the multiple data inputs comprise real time patient diagnostic information.

3. The real time, multiple input display of claim 1, wherein the multiple data inputs comprise previously recorded or web-based material.

4. The real time, multiple input display of claim 1, wherein the display size of data inputs shown within the multiple screen sectors is variable.

5. The real time, multiple input display of claim 1, wherein at least one of the multiple medical inputs may be input via voice-command.

6. The real time, multiple input display of claim 1, wherein the multiple data inputs include, at least, a side-by-side comparison of real-time ultrasound scanning images and an instruction input.

7. The real time, multiple input display of claim 6, wherein the instructional input is an instruction video displaying property ultrasound scanning technique.

8. The real time, multiple input display of claim 1, wherein data inputs from extraneous devices may be mirrored on at least one screen sector of the multi-sector matrix.

9. The real time, multiple input display of claim 1, wherein the multiple data inputs include real time medical diagnostic analysis of a patient shown simultaneously with previously obtained diagnostic information of the patient.

10. The real time, multiple input display of claim 1, wherein display speed of the multiple data inputs on the multiple screen sectors is variable.

11. The real time, multiple input display of claim 1, wherein the multiple data inputs displayed are recorded by the system.

12. The real time, multiple input display of claim 1, wherein the system comprises parametric speakers.

13. The real time, multiple input display of claim 1, wherein control of a cursor associated with the display can be transferred from one user to another user.

14. The real time, multiple input display of claim 1, wherein an instructor's oral presentation of information is shown as scrolling text on the display.

15. The real time, multiple input display of claim 14, wherein the instructor's oral presentation is translated into at least one other language and this language is displayed as scrolling text on the display.

16. The real time, multiple input display of claim 1, wherein artificial intelligence (AI) is utilized for image and clinical data interpretation of the multiple data inputs.

17. The real time, multiple input display of claim 16, wherein clinical data from two or more real time, synchronized inputs is interpreted with or without AI.

18. The real time, multiple input display of claim 16, wherein presentation of the AI clinical data interpretation on the display is inactivated to allow for analysis prior to the AI interpretation.

19. The real time, multiple input display of claim 1, wherein audience responses are displayed in at least one multiple screen sector.

20. The real time, multiple input display of claim 1, wherein remote access to the display information is provided.

21. The real time, multiple input display of claim 1, wherein input and control of the display may be via remote control.

22. The real time, multiple input display of claim 1, wherein users may access a frequently asked questions directory via displaying the frequently asked questions directory on the display.

23. A system for clinical analysis comprising:
a multi-sector matrix screen further comprising multiple screen sectors;
digital recording means to record all information shown on the multiple screen sectors;
at least one parametric speaker;
at least one camera;
at least one sound recording device; and
wherein the multi-sector matrix screen is configured to display multiple data inputs on the multiple screen sectors simultaneously.
